(19) United States
(12) Plant Patent Application Publication
(10) Pub. No.: US 2026/0020508 P1
(43) Pub. Date: Jan. 15, 2026

(54) PETUNIA-CALIBRACHOA HYBRID PLANT NAMED ‘KLECA22835’

(50) ;

(71) Applicant: Klemm+Sohn & Co. KG, Stuttgart (DE)

(72) Inventor: Anita STÖVER, Ostfildern (DE)

(73) Assignee: Klemm+Sohn & Co. KG, Stuttgart (DE)

(21) Appl. No.: 18/768,267

(22) Filed: Jul. 10, 2024

(51) Int. Cl. A01H 6/82; A01H 5/02

(52) U.S. Cl. CPC A01H 6/824; A01H 5/02
USPC PLT/356.11

(57) ABSTRACT

A new Petchoa plant particularly distinguished by having large red-purple flowers and a semi-upright habit, is disclosed.

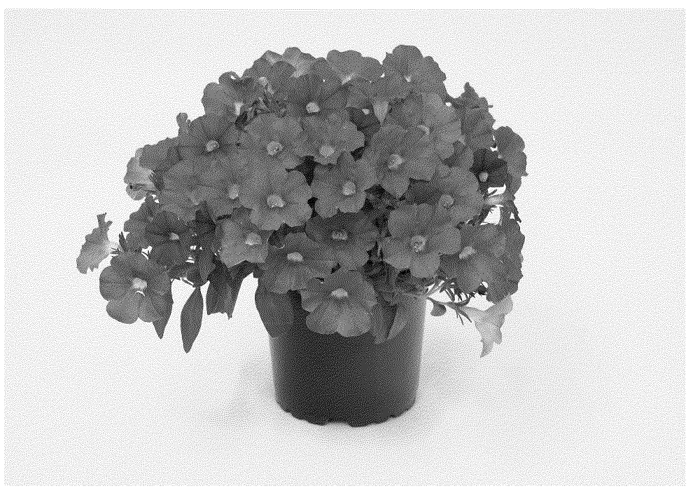

Genus and species: Petunia x Calibrachoa hybrid.

Variety denomination: ‘KLECA22835’.

### BACKGROUND OF THE NEW PLANT

The present invention comprises a new and distinct variety of Petunia x Calibrachoa hybrid, commonly known as Petchoa, and hereinafter referred to by the variety name ‘KLECA22835’. ‘KLECA22835’ originated from a controlled-cross between the female variety SuperCal® Cherry Improved (also known as ‘SAKPXC012’, U.S. Plant Pat. No. 26,332) and the male Calibrachoa variety ‘KLECA17395’ (unpatented).

In August 2017, the female variety SuperCal® Cherry Improved and the male variety ‘KLECA17395’ were crossed via a controlled-cross in Stuttgart, Germany and seeds were obtained. The seeds were sown and plants were grown for evaluation. A plant line was selected in May 2018 and subsequently named ‘KLECA22835’. ‘KLECA22835’ was first vegetatively propagated by cuttings in Stuttgart, Germany in May 2018. ‘KLECA22835’ was found to reproduce true to type in successive generations of asexual propagation via cuttings.

### SUMMARY

The following are the most outstanding and distinguishing characteristics of this new variety when grown under normal horticultural practices in Stuttgart, Germany.
1. Large, red-purple flowers; and
2. Semi-upright plant habit.

### DESCRIPTION OF THE PHOTOGRAPH

This new Petchoa plant is illustrated by the accompanying photograph which shows a close-up of the whole plant.

The photograph is of a 12-week-old-plant grown indoors in Stuttgart, Germany in May 2022.

The colors shown are as true as can be reasonably obtained by conventional photographic procedures.

### DESCRIPTION OF THE NEW VARIETY

The following detailed descriptions set forth the distinctive characteristics of ‘KLECA22835’. The data which define these characteristics were collected from asexual reproductions carried out in Stuttgart, Germany. Data was collected on 8-week old plants after transplant in Stuttgart, Germany. Color references are to The R.H.S. Colour Chart of The Royal Horticultural Society of London (R.H.S.), 5^{th} edition (2007).
Classification:
   Family.—Solanaceae.
   Species.—Petunia x Calibrachoa hybrid.
   Common name.—Petchoa.
Parentage:
   Female.—SuperCal® Cherry Improved (also known as ‘SAKPXC012’, U.S. Plant Pat. No. 26,332).
   Male.—‘KLECA17395’ (unpatented).
Plant description:
   Life cycle.—Annual.
   Form.—Short lived subshrub.
   Habit.—Semi-upright.
   Height (from soil line to top of foliage).—20.0 cm.
   Spread.—45.0 cm.
Propagation:
   Type cuttings.—Tip cuttings.
   Time to produce a rooted cutting.—3 weeks.
Stems:
   Stem color.—RHS 144B.
   Pubescence.—Present.
   Pubescence color.—RHS 145C.
   Stem diameter.—0.3 cm.
   Stem length.—From soil line to first node: 0.8 cm. Total length: 29.0 cm.
   Internode length.—3.5 cm.
Leaves:
   Arrangement.—Alternate.
   Shape.—Acute elliptica.
   Apex.—Acute.
   Base.—Attenuate.
   Margin.—Entire.
   Surface texture.—Smooth.
   Surface pubescence.—Absent.
   Venation.—Pinnate.
   Venation color.—Upper surface: RHS 146C. Lower surface: RHS 146D.
   Length.—3.0 cm.
   Width.—0.9 cm.
   Color.—Upper surface: RHS 137A. Lower surface: RHS 137C.
   Fragrance.—Absent.
Flowers:
   Flowering habit.—Continuous.
   Flower type.—Single.
   Flowering requirements.—Natural sunlight.
   Duration of flowers.—7 days.
   Corolla texture (both surfaces).—Smooth.
   Corolla.—Rotate.
   Shape.—Funnel form.
   Fragrance.—Absent.
   Number of flowers per plant.—28.
Flower buds (before opening):
   Surface.—Smooth, dull.
   Length.—3.0 cm
   Diameter.—0.4 cm.
   Shape.—Irregular to oblong.
   Color.—RHS N77C.
   Typical number per plant.—Approximately 15 to 20.
Peduncle:
   Length.—3.3 cm.
   Diameter.—0.1 cm.
   Color.—RHS 145A.
   Texture.—Smooth.
   Anthocyanin color.—Absent.
Flower description:
   Flower depth.—4.5 cm.
   Flower tube length.—2.5 cm.
   Flower tube diameter.—0.9 cm.
   Flower diameter.—5.4 cm.
   Flower throat diameter.—1.4 cm.
   Pedicel color.—RHS 145A.
   Calyx.—RHS 146C.
Sepals:
   Quantity.—5.
   Shape.—Lanceolate.
   Texture.—Smooth.
   Apex.—Acute.
   Margin.—Entire.
   Length.—2.0 cm.
   Diameter.—0.3 cm.
   Sepal color.—Upper surface: RHS N137A. Lower surface: RHS 137C.
Petals:
   Shape.—Reniform.
   Quantity.—5, fused.
   Length.—2.5 cm.
   Apex.—Obtuse.
   Margin.—Entire.
   Base.—Wide open fused.
   Pubescence.—Absent.
   Lobe length.—2.2 cm.
   Lobe width.—2.5 cm.
   Color.—Lobe color: Upper surface: RHS N74A. Lower surface: RHS N74D. Corolla tube color: Inner: RHS 9B. Outer: RHS 9D.
   Fragrance.—Absent.
   Lobing intensity.—Low.
   Vein intensity.—Low.
Reproductive organs:
   Stamen.—Quantity: 5. Anther color: RHS 10A. Anther length: 0.2 cm. Filament length: 0.9 cm. Filament color: RHS 10D. Pollen amount: Moderate. Pollen color: RHS 10B.
   Pistil.—Quantity: 1. Length: 1.7 cm. Ovary: Superior. Placenta arrangement: Free, central. Stigma color: RHS 144A. Stigma shape: Rounded. Style length: 1.4 cm. Style color: RHS 145B.
Fruit/seed set: Very low.
Disease/insect resistance: None observed.

### COMPARISON WITH PARENTAL LINES AND KNOWN VARIETY

‘KLECA22835’ is distinguished from its parents as described in Table 1:

TABLE 1
Comparison with Parental Lines

| | | Female parent | |
|---|---|---|---|
| | | SuperCal ® | |
| | Variety | Cherry | Male parent |
| Trait | ‘KLECA22835’ | Improved | ‘KLECA17395’ |
| Flower diameter | 5.4 cm | 4.7 cm | 3.9 cm |
| Flower color | Red-purple N74A | Red-purple N57 | White |

‘KLECA22835’ is most similar to SuperCal® Blushing Pink (also known as ‘SAKPXC008’, U.S. Plant Pat. No. 25,418). Differences between the two varieties are described in Table 2:

TABLE 2
Comparison with Similar Variety

| | | SuperCal ® |
|---|---|---|
| | Variety | Blushing |
| Trait | ‘KLECA22835’ | Pink |
| Flower color | Red-purple N74A | Light pink |

What is claimed is:

1. A new and distinct Petunia-Calibrachoa hybrid plant named ‘KLECA22835’ as shown and described herein.
